(19) United States
(12) Patent Application Publication
(10) Pub. No.: US 2016/0338397 A1
(43) Pub. Date: Nov. 24, 2016

(54) COMPOSITION WITH SYNBIOTICS

(71) Applicant: N.V. Nutricia, Zoetermeer (NL)

(72) Inventors: Jan KNOL, Utrecht (NL); Bernd STAHL, Utrecht (NL)

(73) Assignee: N.V. Nutricia, Zoetermeer (NL)

(21) Appl. No.: 15/080,913

(22) Filed: Mar. 25, 2016

Related U.S. Application Data
(63) Continuation of application No. 14/175,585, filed on Feb. 7, 2014, now abandoned.
Application No. 14/175,585 is a continuation of application No. 12/743,806, filed on Sep. 8, 2010, now abandoned.

(30) Foreign Application Priority Data
Nov. 20, 2007 (NL) PCT/NL2007/050575

(51) Int. Cl. A23L 33/135; A23L 33/17; A61K 35/745; A23L 33/21; A61K 35/747; A23L 33/00; A23L 33/125

(52) U.S. Cl. CPC A23L 33/135; A23L 33/30; A23L 33/40; A23L 33/17; A23L 33/125; A23L 33/21; A61K 35/747; A61K 35/745; A23V 2002/00; A61K 2035/115

(57) ABSTRACT

Nutritional compositions with health benefits comprising bacterial strains and galactooligosaccharides are disclosed.

### CROSS REFERENCE TO RELATED APPLICATIONS

This is a Continuation application of U.S. application Ser. No. 14/175,585, filed Feb. 2, 2014, which is a Continuation application of U.S. application Ser. No. 12/743,806, filed Sep. 8, 2010 (now abandoned), which is a National Stage Application of PCT/EP2008/065936, filed Nov. 20, 2008, which claims priority to International Application No. PCT/NL2007/050575, filed Nov. 20, 2007. The foregoing application is incorporated by reference herein in its entirety.

### FIELD OF THE INVENTION

The present invention relates to nutritional compositions with health benefits.

### BACKGROUND OF THE INVENTION

WO 2007/054208 describes an edible product containing probiotic bacteria in an amount of at least 10³ bacteria per gram, and at least 0.5 mg/g of ginseng polysaccharides containing at least 2 monosaccharide units, preferably at least 4 monosaccharide units. Furthermore the use of the aforementioned product in therapeutic and prophylactic treatments is described.

U.S. 2002/044926 describes methods and compositions for the oral administration of at least one Lactobacillus and/or other probiotic organisms, such as Bifidobacterium, for improving vaginal health. The document also discloses methods and compositions to treat vaginitis, bacterial vaginosis and reduce Candida colonization.

### SUMMARY OF THE INVENTION

A main problem with oral administration of probiotic bacteria is an insufficient or bad colonization of said probiotic bacteria in the intestinal tract. The bad colonization has as a consequence that the dosage of probiotic bacteria has to be increased and/or a more frequent administration is needed. This is both costly, leads to undesirable frequency of intake and/or decreases the occurrence of health benefits.

The present inventors surprisingly found that probiotics Lactobacillus strain DN-114 001 and/or Bifidobacterium strain DN-173 010 show an improved growth and/or colonization when co-administered with galactooligosaccharides. The inventors believe that the galactooligosaccharides increase the concentration of acetate in the intestinal tract, creating favorable growth conditions for DN-114 001 and/or DN-173 010. It was found that galactooligosaccharides, preferably in combination with fructooligosaccharides (e.g. inulin) stimulate the colonization (e.g. growth) of DN-114 001 and/or DN-173 010 under conditions mimicking the in vivo situation.

Without being bound by theory, it is the inventors believe that this improvement is due to the stimulated in vivo production of acetate when galactooligosaccharides are (co-) administered with the probiotic strains. Oral administration of galactooligosaccharides, preferably combined with fructooligosaccharides results in acetate production in the intestinal tract

It was also found that growth of DN-114 001 and/or DN-173 010 was inhibited by a high pH. The pH sensitivity results in a reduced growth in the intestinal tract when DN-114 001 and/or DN-173 010 are included in (high) protein containing compositions. Orally ingested proteins often are not completely absorbed (particularly in infants and elderly) resulting in protein reaching the lower parts of the intestinal tract (e.g. the colon). In the colon the proteins are fermented resulting in ammonia production, increasing the pH. This effect can be counteracted by administering galactooligosaccharides, preferably combined with fructooligosaccharides. As the administration of galactooligosaccharides effectively reduces, and/or prevents a rise of, the intestinal pH, this is a (further) mechanism by which colonization of DN-114 001 and/or DN-173 010 is stimulated by galactooligosaccharides, preferably combined with one or more other oligosaccharides. Furthermore, survival and/or colonization of the present strains is promoted by suppressing intestinal bacteria and/or reducing growth of intestinal bacteria and/or reducing or preventing adhesion of intestinal bacteria, particularly by co-administration of galacturonic acid oligosaccharides, e.g. pectin degradation product.

Furthermore the present inventors found that high protein intake can be an important cause of intestinal disbalance. This is particularly the case for subjects having an impaired intestinal protein metabolism such as infants and elderly. Nevertheless, protein intake, preferably in high amounts, is particularly desired for growth in case of infants and prevention of catabolism in the case of elderly. However, due to the high protein intake, the protein may not be fully digested and absorbed, resulting in protein reaching the small intestine and colon. Here the protein has the effect of stimulating growth of e.g. Clostridium and disbalancing the flora and potentially resulting in infections.

A further aim of the present invention is to stimulate the growth and development of a healthy intestinal flora when DN-114 001 is orally administered in a protein containing formulation to subjects suffering or potentially suffering from an impaired immune system. This is accomplished by oral (co) administration of galactooligosaccharides.

### DETAILED DESCRIPTION

Thus the present invention concerns the use of a protein containing composition comprising

a. the bacterial strain identified as DN-114 001 (CNCM 1-1518) and/or the bacterial strain identified as DN-173 010 (CNCM 1-2494); and

b. 0.1 to 95 g of (non-digestible) galacto-oligosaccharides per 100 g dry weight; for (i) the treatment and/or prevention of infections and/or (ii) stimulating the immune system, wherein the composition has an osmolality between 50 and 500 mOsm/kg.

Also the present invention concerns a protein containing composition comprising

a. strain DN-114 001 (CNCM 1-1518) and/or strain DN-173 010 (CNCM 1-2494); and

b. galactooligosaccharides.

### Strains

The present composition comprises live or dead bacteria from the strain DN-114 001 and/or DN-173 010. As described in WO 2006/077171, the strain DN-114 001 has been deposited at the Collection Nationale de Cultures de Microorganisms (CNCM, Institut

Pasteur, Paris, France) under the number I-1518. This strain is sometimes designated as Lactobacillus casei. It is (commercially) identified as DN-114 001. DN-173 010 also has been deposited at the Collection Nationale de Cultures de Microorganisms (CNCM, Institut Pasteur, Paris, France) and is registered under the number CNCM I-2494 and is sometimes designated as Bifidobacterium animalis. DN-114 001 is available in Actimel™ from Danone. DN-173 010 is available in Activia™ from Danone.

The present composition preferably comprises 10² to 10¹³ colony forming units (cfu) of bacteria per gram (g) dry weight of the present composition, preferably 10² to 10¹² cfu, more preferably 10⁵ to 10¹⁰ cfu, most preferably from 10⁴ to 5×10⁹ cfu. The present composition preferably comprises 10² to 10¹³ colony forming units (cfu) of DN-114 001 and/or DN-173 010 per gram (g) dry weight of the present composition, preferably 10² to 10¹² cfu, more preferably 10⁵ to 10¹⁰ cfu, most preferably from 10⁴ to 5x10⁹ cfu of DN-114 001 and/or DN-173 010 per g dry weight.

### Galactooligosaccharides

The present inventors found that galacto-oligosaccharides can be advantageously used in the present composition, because these oligosaccharides where particularly effective in stimulating the growth of Bifidobacteria and/or DN-114 001. The present composition preferably comprises galactooligosaccharides which are fermented into acetate. The term “galacto-oligosaccharide” as used herein refers to a non-digestible oligosaccharide, wherein at least 30% of the saccharide units are galactose units, preferably at least 50%, more preferably at least 60%. Lactose is considered digestible. The present composition preferably comprises galacto-oligosaccharides with a DP of 2 to 100, more preferably a DP of 2 to 10. Preferably the saccharides of the galacto-oligosaccharide are (3-linked, as is the case in human milk oligosaccharide-core structures.

Preferably the present composition comprises a galacto-oligosaccharide selected from the group consisting of (trans)galacto-oligosaccharides, lacto-N-tetraose (LNT) and lacto-N-neotetraose (neo-LNT). In a particularly preferred embodiment the present composition comprises transgalacto-oligosaccharide. Transgalacto-oligosaccharide can be defined as [galactose]ₙglucose and/or [galactose]ₙglucose-([galactose]m) wherein n and m are integers from 1 up to and including 60, i.e. 2, 3, 4, 5, 6, . . . , 59, 60; preferably n is 2, 3, 4, 5, 6, 7, 8, 9 and/or 10. Preferably m is 2, 3, 4, 5, 6, 7, 8, 9 and/or 10. Preferably the present composition comprises [galactose]ₙglucose wherein n is an integer from 1 up to and including 60. Preferably n is 2, 3, 4, 5, 6, 7, 8, 9 and/or 10. Transgalacto-oligosaccharides (TOS) are for example sold under the trademark Vivinal™ (Borculo Domo Ingredients, Netherlands) and Oligomate 55 from Yakult. Preferably the saccharides of the galacto-oligosaccharides are mainly β-linked.

The present composition preferably comprises 0.1 to 95 g of the galacto-oligosaccharides per 100 g dry weight, preferably between 0.5 and 50 g, more preferably between 1 and 25 g, most preferably between 2 and 10 g. The present composition preferably comprises 0.5 to 75 g non-digestible oligosaccharides per 100 g dry weight of the present composition, preferably between 1 and 50 g, more preferably between 2 and 25 g. The present method preferably comprises the administration of a serving comprising between 0.05 and 25 g galacto-oligosaccharide, preferably between 0.1 and 5 g. The present method preferably comprises the administration of a serving comprising between 0.05 and 25 g galactooligosaccharides, preferably between 0.1 and 5 g galacto-oligosaccharides.

### Mixture of (Prebiotic) Oligosaccharides

The present composition preferably comprises at least two non-digestible neutral oligosaccharides with different average degrees of polymerization (DP). The present inventors have found that combinations of oligosaccharides can improve the acetate production and/or have an improved pH lowering effect, resulting in an improved colonization of the intestinal tract by DN-114 001 and/or DN-173 010. In the context of this invention neutral oligosaccharide is different from galacturonic acid oligosaccharide as defined hereinbelow.

The present composition preferably comprises two non-digestible neutral oligosaccharides with a different structure. The present composition preferably comprises at least two different non-digestible neutral oligosaccharides, wherein the non-digestible oligosaccharides have a homology in saccharide units below about 90%, preferably below 50%, even more preferably below 25%, even more preferably below 5%. The term “homology” as used in the present invention is the cumulative of the percentage of same saccharide unit in the different non-digestible oligosaccharides. For example, oligosaccharide 1 (OL1) has the structure fruc-fruc-glu-gal, and thus comprises 50% fruc, 25% gal and 25% glu. Oligosaccharide 2 (OL2) has the structure fruc-fruc-glu, and thus comprises 66% fruc, 33% glu. The different non-digestible oligosaccharides thus have a homology of 75% (50% fruc +25% glu).

Preferably the present composition comprises, besides galactooligosaccharides, at least one non-digestible oligosaccharide selected from the group consisting of fructo-oligosaccharides (including inulins), non-digestible dextrins, xylo-oligosaccharides, arabino-oligosaccharides, arabinogalacto-oligosaccharides, gluco-oligosaccharides (including cyclodextrins and gentio-oligosaccharides), chito-oligosaccharides, glucomanno-oligosaccharides, galactomanno-oligosaccharides, mannan-oligosaccharides, fuco-oligosaccharides, galacturonic acid oligosaccharides, guluronic acid oligosaccharides, mannuronic acid oligosaccharides, iduronic acid oligosaccharides, riburonic acid oligosaccharides, glucuronic acid oligosaccharides and mixtures thereof, more preferably the present composition comprises, besides galactooligosaccharides, at least one non-digestible oligosaccharide selected from the group consisting of fructo-oligosaccharides, galactooligosaccharides (including transgalacto-oligosaccharides), fuco-oligosaccharides (including sulphated fucoidan oligosaccharides) and galacturonic acid oligosaccharides and mixtures thereof, even more preferably the present composition comprises galactooligosaccharides and fructooligosaccharides and/or inulin.

Preferably the present composition contains sialic acid, 3-sialyllactose, 6-sialyllactose, 2-fucosyllactose, 3-fucosyllactose and/or lactosylsialyltetrasaccharides.

The present composition preferably comprises galacto-oligosaccharides and fructo-oligosaccharides, more preferably transgalacto-oligosaccharides with a DP of 2-7 and fructo-oligosaccharides with a DP of 2-100. The combination of galactooligosaccharides and fructooligosaccharides improves colonization of the DN-114 001 and/or DN-173 010. The present composition preferably comprises fructo-oligosaccharides (e.g. inulin). Preferably at least 50 wt. % of the non-digestible oligosaccharides in the present composition have a degree of polymerization of 2 to 60. In a particular preferred embodiment the present composition comprises at least galacto-oligosaccharides and fructo-oligosaccharides. The galacto-oligosaccharides preferably comprise saccharides with a DP of 2 to 10. The fructo-oligosaccharides preferably comprise saccharides with a DP of 2 to 100, preferably a DP between 5 and 100. Preferably, the galacto-oligosaccharide comprises beta bonds, as is the case in human milk oligosaccharides.

Preferably the present composition contains neutral non-digestible oligosaccharides with the following weight ratios:

a. (non-digestible neutral oligosaccharides with DP 2 to 5):(non-digestible neutral oligosaccharides with DP 6, 7, 8, and/or 9)>1; and/or

b. (non-digestible neutral oligosaccharides with DP 10 to 60):(non-digestible neutral oligosaccharides with DP 6, 7, 8, and/or 9)>1.

Preferably both weight ratios are above 2, even more preferably above 5.

In a preferred embodiment the present composition comprises galacturonic acid oligosaccharides. The galacturonic acid oligosaccharides of the invention advantageously reduce the adhesion of pathogenic micro-organisms to the intestinal epithelial cells, thereby reducing colonization of (nosocomial) pathogenic bacteria and/or improves barrier integrity of the in the colon. Furthermore, the galacturonic acid oligosaccharides of the present invention stimulate the formation of a healthy intestinal flora and may are fermented, resulting in a production of intestinal organic acids and a reduction of intestinal pH, which inhibit the growth of pathogenic bacteria. The co-administration of galacturonic acid oligosaccharides therefore further improves protection from infections due to the underdeveloped barrier function and/or underdeveloped intestinal bacterial flora. Preferably to present composition comprises a pectin degradation product, preferably with a DP between 2 and 250.

The term galacturonic acid oligosaccharide as used in the present invention preferably refers to an oligosaccharide wherein at least 50% of the monosaccharide units present in the oligosaccharide are galacturonic acid. The present composition preferably comprises galacturonic acid oligosaccharide with a DP between 2 and 250, preferably 2 and 50, more preferably 2 and 20. The present composition preferably comprises galacturonic acid oligosaccharide a mass at peak of a curve determined by SEC/GPS of between DP 2 and DP 500, preferably between DP 2 and 200.

The galacturonic acid oligosaccharides used in the invention are preferably prepared from pectin, pectate, and/or polygalacturonic acid. The galacturonic acid oligosaccharides used in the invention are preferably prepared from fruit vegetable and herbal plants used for human nutrition. The galacturonic acid oligosaccharide is preferably derived from pectin. Preferably the pectin oligosaccharide is prepared by hydrolysis and/or beta-elimination of fruit pectin and/or vegetable pectin, more preferably from apple, citrus and/or sugar beet pectin, more preferably the apple, citrus and/or sugar beet pectin has been treated by at least a lyase. Preferably the pectin lysate and/or the galacturonic acid oligosaccharide is prepared from bacterial production.

In a preferred embodiment, at least one of the terminal hexuronic acid units of the galacturonic acid oligosaccharide has a double bond, which is preferably situated between the C₄ and C₅ position of the terminal hexuronic acid unit. The double bond effectively protects against attachment of pathogenic bacteria to intestinal epithelial cells. This is advantageous for infants delivered by caesarean section. Preferably at least 5%, more preferably at least 10%, even more preferably at least 25% of the terminal hexuronic acid units of the galacturonic acid oligosaccharide is an unsaturated hexuronic acid unit. As each individual galacturonic acid oligosaccharide preferably comprises only one unsaturated terminal hexuronic acid unit, preferably less than 50% of the terminal hexuronic acid units is an unsaturated hexuronic acid unit, i.e. comprises a double bond.

The present composition preferably comprises between 0.01 and 10 g galacturonic acid oligosaccharide with a DP of 2 to 250 per 100 g dry weight of the composition, more preferably between 0.05 and 6 g, even more preferably 0.2 to 2 g. Preferably the present composition comprises between 0.01 and 10 g galacturonic acid oligosaccharide with a DP of 2 to 25 per 100 g dry weight of the nutritional composition, more preferably between 0.05 and 6 g, even more preferably 0.2 to 2 g. The short (DP 2 to 25) chain galacturonic acid oligosaccharides are more effective and/or better suitable for inclusion in the present composition. In one embodiment the present composition, besides galacto-oligosaccharide, further comprises a saccharide selected from the group consisting of inulin, fructooligosaccharides and galacturonic acid oligosaccharide. In one embodiment the present composition comprises (i) galacto-oligosaccharide, (ii) inulin and/or fructooligosaccharides and (iii) a pectin degradation product.

### Bacteria

In a further improvement, the present composition contains multiple probiotic bacteria. By the improved diversity, the barrier integrity is stimulated and/or a healthy flora is better maintained. Additionally also these bacteria benefit from the co-administered galactooligosaccharides, preferably including the additional neutral and/or galacturonic acid oligosaccharides, thereby improving survival and intestinal flora.

Preferably the present composition comprises bacteria of the genus Lactobacillus and/or Bifidobacterium. Preferably the composition additionally comprises at least one Bifidobacterium selected from the group consisting of B. longum, B. breve, B. infantis, B. catenulatum, B. pseudocatenulatum, B. adolescentis, B. animalis, B. gallicum, B. lactis and B. bifidum, more preferably at least one Bifidobacterium selected from the group consisting of B. breve, B. infantis, B. bifidum, B. catenulatum, B. longum, even more preferably at least one Bifidobacterium selected from the group consisting of B. breve and B. longum, most preferably B. breve.

Preferably the present composition additionally comprises a Lactobacillus selected from the group consisting of L. casei, L. reuteri, L paracasei, L. rhamnosus, L. acidophilus, L. johnsonii, L. lactis, L. salivarius, L. crispatus, L. gasseri, L. zeae, L. fermentum and L. plantarum.

Preferably the present composition comprises Lactobacillus bulgaricus and/or Streptococcus thermophilus.

The present composition preferably comprises 10² to 10¹³ colony forming units (cfu) of (lactic acid producing) bacteria per g dry weight of the present composition, preferably 10² to 10¹² cfu, more preferably 10⁵ to 10¹⁰ cfu, most preferably from 10⁴ to 5×10⁹ cfu.

### Protein

The present composition preferably contains protein. The protein used in the present composition preferably comprises at least one selected from the group consisting of non-human animal proteins (such as milk proteins, meat proteins and egg proteins), vegetable proteins (such as soy protein, wheat protein, rice protein, potato protein and pea protein), free amino acids and mixtures thereof. Cow's milk derived nitrogen source, particularly cow milk protein proteins such as casein and whey proteins are particularly preferred. More preferably the composition is fermented with bacteria. Preferably the protein component comprises intact proteins, more preferably intact bovine whey proteins and/or intact bovine casein proteins.

The present composition, when in liquid form, preferably comprises between 0.5 and 8 g protein per 100 ml, preferably comprises between 1 and 8 gram protein per 100 ml, preferably between 1.5 and 6 g protein per 100 ml, more preferably between 1.5 and 3 g per 100 ml.

When the composition is administered to an infant, the composition preferably contains sweet whey and/or acid whey.

### Long-Chain Polyunsaturated Fatty Acids

The effectiveness of the present symbiotic composition can be enhanced by including LC-PUFA and/or nucleotides in the present composition, as co-administration of the non-digestible oligosaccharides with the LC-PUFA and/or nucleotides causes a delay in absorption of the LC-PUFA and/or nucleotides in the small intestine, thereby prolonging and/or increasing the effects of the LC-PUFA and/or nucleotides in the colon.

Preferably the present composition comprises at least one LC-PUFA selected from the group consisting of eicosapentaenoic acid (EPA, 20:5 n3), docosahexaenoic acid (DHA, 22:6 n3), arachidonic acid (ARA, 20:4 n6) and docosapentaenoic acid (DPA, 22:5 n3).

The LC-PUFA may be provided as free fatty acids, in triglyceride form, in diglyceride form, in monoglyceride form, in phospholipid form, or as a mixture of one of more of the above, preferably in triglyceride form. The present composition preferably comprises at least one of ARA and DHA in phospholipid form.

### Nucleotides

In a preferred embodiment the present composition comprises nucleotide and/or nucleotide precursors selected from the group consisting of nucleoside, purine base, pyridine base, ribose and deoxyribose. More preferably the composition comprises nucleotide. The nucleotide is preferably in the monophosphate, diphosphate or triphosphate form, more preferably a nucleotide monophosphate. The nucleotide preferably is a ribonucleotide or a deoxyribonucleotide, more preferably a ribonucleotide. The nucleotides can be monomeric, dimeric or polymeric (including RNA and DNA). The nucleotides preferably are present as a free acid or in the form of a salt, more preferably monosodium salt. Incorporation of nucleotide in the present composition improves intestinal barrier integrity and/or maturation.

Preferably the composition comprises 5 mg to 5 g, more preferably 5 to 1000 mg, most preferably 10 to 500 mg nucleotides per 100 g dry weight of the present composition. The nucleotides further stimulate the immune system thereby enhancing protection against a high load of intestinal pathogens such as E. coli.

### Formulae

The present composition is preferably enterally administered, more preferably orally.

In one embodiment the present composition is an infant formula. The present composition is preferably a synthetic formula, prepared by admixing different ingredients. The present composition is not a naturally (non-treated) occurring mammalian milk, e.g. not human breast milk. The present composition can be advantageously used as a complete nutrition for infants. The present composition preferably comprises lipid, protein and carbohydrate and is preferably administered as a liquid food. The term “liquid food” as used in the present invention includes dry food (e.g. powders) which are accompanied with instructions so as to admix said dry food mixture with a suitable liquid, e.g. water.

The present composition preferably provides nutrition and comprises a lipid component, a protein component and a carbohydrate component. The lipid component preferably provides 5 to 50% of the total calories, the protein component preferably provides 5 to 50% of the total calories, and the carbohydrate component preferably provides 15 to 90% of the total calories. The present composition is preferably used to provide nutrition to an infant, e.g. as an infant formula, wherein the lipid component provides 35 to 50% of the total calories, the protein component provides 7.5 to 12.5% of the total calories, and the carbohydrate component provides 40 to 55% of the total calories. For calculation of the % of total calories for the protein component, the total of energy provided by the proteins, peptides and amino acids needs to be taken and the energy provided by digestible carbohydrates.

In a further embodiment, the present composition is suitable for administration to an elderly person and/or a sick person. An elderly person is typically an adult having an age of 55 years and above. Hospitalized adults will particularly benefit from the present composition. Still, healthy adults can also advantageously use the present products. Healthy adults can improve resistance to infections by ingesting the present composition. When the present composition is preferably used as a nutritional composition for adults, in particular for providing nutrition preferably to elderly and hospitalized adults, the lipid component preferably provides 20 to 50% of the total calories, the protein component provides 10 to 35% of the total calories, and the carbohydrate component provides 30 to 75% of the total calories.

The present composition preferably comprises carbohydrates. Preferably the composition comprises digestible carbohydrates. The digestible carbohydrates used in the present composition are preferably selected from the group consisting of sucrose, lactose, glucose, fructose, corn syrup solids, starch and maltodextrins, and mixtures thereof, more preferably lactose.

The present composition preferably comprises lipid. Preferably the present composition comprises a combination of at least one lipid selected from the group consisting of vegetable lipids and animal lipids and at least one oil selected from the group consisting of fish, animal, vegetable, algae, fungal and bacterial oil.

### Stool Irregularities

Reduction of stool irregularities (e.g. hard stools, insufficient stool volume, diarrhea) is a main aim of the present composition. Hence the present composition is preferably used in a method for the prevention and/or treatment of diarrhea, constipation and/or bloating. In order to prevent intestinal discomfort, it is important that the present ingredients are administered in a composition comprising an osmolality between 50 and 500 mOsm/kg. Hence the present composition preferably has an osmolality between 50 and 500 mOsm/kg, more preferably between 100 and 400 mOsm/kg. In view of the above, it is also important that the liquid food does not have an excessive caloric density, however still provides sufficient calories to feed the subject. Hence, the liquid food preferably has a caloric density between 0.1 and 2.5 kcal/ml, even more preferably a caloric density of between 0.5 and 1.5 kcal/ml, most preferably between 0.6 and 0.8 kcal/ml.

To reduce stool problem, the daily dosage volume of the present product is preferably limited. Hence the present composition is preferably administered in a volume between 25 and 200 ml/day, preferably between 75 and 150 ml/day. This preferably applies to nutritional compositions for adults.

### Application

The present composition is particularly suitable for the treatment or prevention of infection, allergy or diarrhea. Furthermore, the present invention is particularly suitable for subjects where the intestinal flora is absent or severely disturbed. Hence the present invention also provides the use of the present composition for administration to infants born by caesarean section, preferably to stimulate development of the intestinal flora. In a further aspect, the present invention provides the use of the present composition to be administered to subjects having completed an antibiotic treatment, preferably to restore the intestinal flora, preferably for recovery after antibiotics treatment. The present composition is particularly suitable for stimulating gut health, particularly in women.

### EXAMPLES

### Example 1

### Acetate Production

Micro-organisms were obtained from fresh faeces from bottle fed babies. As substrate either prebiotics a) transgalacto-oligosaccharides (TOS), b) inulin HP and c) TOS and inulin HP mixture in a 9/1 (w/w) ratio was used. A volume of 3.0 ml of the faecal suspension was combined with 85 mg prebiotics in a bottle and mix thoroughly. At t=0 and t=3 a sample was withdrawn.

In vitro fermentation was carried out using the following samples: a) 85 mg TOS; b) 85 mg inulin HP; c) 85 mg TOS/inulin HP with a ratio of TOS/inulin HP of 9/1 (w/w).

A good acetate production was observed for a) TOS (0.23 mmol/gram fiber), while b) inulin did not show acetate production after 3 hours (0 mmol/g fiber). A synergistic higher formation of acetate is observed for the mixture of two different oligosaccharides c) TOS/inulin HP (0.4 mmol/gram fiber) compared to the single components a) TOS and b) inulin HP.

### Example 2

### Composition with Protein

Liquid composition comprising per 100 ml:
0.7 g TOS,
0.1 g inulin HP,
1.6 g whey protein,
7 g lactose
1.5 g fat,
vitamins and minerals
10⁶ cfu DN-114 001 and 10⁵ cfu DN-173 010.

### Example 3

### Composition with Protein

Composition with protein comprising
yoghurt,
skimmed milk,
TOS and oligofructose,
dextrose,
pectin,
modified tapioca starch,
flavouring,
aspartame,
acesulfame K and
DN-114 001.

1. A food composition, comprising:
a. a lipid component which provides 35 to 50% of the total calories,
b. a protein component which provides 7.5 to 12.5% of the total calories,
c. a carbohydrate component which provides 40 to 55% of the total calories, and
d. Lactobacillus strain DN-114 001 (CNCM 1-1518), Bifidobacterium strain DN-173 010 (CNCM 1-2494) or both; and
e. galactooligosaccharides.

2. The food composition according to claim 1, further comprising a saccharide selected from the group consisting of inulin and fructooligosaccharides.

3. The food composition according to claim 1, further comprising a galacturonic acid oligosaccharide.

4. The food composition according to claim 3, wherein the galacturonic acid oligosaccharide is a pectin degradation product.

5. The food composition according to claim 1, wherein at least 60% of the saccharide units in the galactooligosaccharides are galactose units.

6. The food composition according to claim 1, wherein the galactooligosaccharides have a degree of polymerization (DP) of 2 to 10.

7. The food composition according to claim 1, wherein the saccharides of the galactooligosaccharides are mainly β-linked.

8. The food composition according to claim 1, which comprises 10² to 10¹³ colony forming units (cfu) of Lactobacillus strain DN-114 001 and Bifidobacterium strain DN-173 010 per gram (g) dry weight.

9. The food composition according to claim 8, which comprises 10⁵ to 10¹⁰ colony forming units (cfu) of Lactobacillus strain DN-114 001 and Bifidobacterium strain DN-173 010 per gram (g) dry weight.

10. The food composition according to claim 1, which, when in liquid form, comprises between 1 and 3 g protein per 100 ml.

11. A method of providing nutrition to an infant, comprising administering to an infant in need thereof a composition according to claim 1.

12. A method of restoring intestinal flora in a subject recovering from antibiotic treatment comprising administering to the subject a composition according to claim 1.
